(19) United States
(12) Patent Application Publication
(10) Pub. No.: US 2015/0313526 A1
(43) Pub. Date: Nov. 5, 2015

(54) SYSTEM AND METHOD FOR BLOOD SAMPLING FAILURE ANALYSIS AND CORRECTION

(71) Applicant: EDWARDS LIFESCIENCES CORPORATION, Irvine, CA (US)

(72) Inventor: Paul S. Van Wieren, Los Angeles, CA (US)

(73) Assignee: Edwards Lifesciences Corporation, Irvine, CA (US)

(21) Appl. No.: 14/646,169

(22) PCT Filed: Nov. 8, 2013
(86) PCT No.: PCT/US13/69103

§ 371 Date: May 20, 2015

Related U.S. Application Data
(60) Provisional application No. 61/736,362, filed on Dec. 12, 2012.

(51) Int. Cl. A61B 5/15; A61B 5/00; A61B 5/1486; A61B 5/157; A61B 5/1495; A61B 5/145

(52) U.S. Cl. CPC A61B 5/150961; A61B 5/1495; A61B 5/14532; A61B 5/1486; A61B 5/150992; A61B 5/157; A61B 5/486; A61B 5/150099; A61B 2560/0475; A61B 2560/0238; A61B 2562/0247; A61B 2560/0487

(57) ABSTRACT

Disclosed herein are systems and methods for analyzing failed bodily fluid sampling. The systems and methods include providing a system having a sampling line and an access device, the access device having an inner space, wherein the sampling line is positioned in the inner space; performing a sampling event; obtaining one or more status values of the sampling event; comparing the one or more status values with at least one predetermined value corresponding to a failed sampling event; and determining whether the sampling event failed based on the comparing step. In the event of sampling failure, the systems and methods provide flushing the sampling line or a portion of the inner space of the access device via the flow controller; detecting pressure; and determining one or more possible root causes for the failure of the sampling event based on the detected pressure.

### BACKGROUND

Currently, some glucose monitoring systems with fluidic systems have the ability to identify failed blood samples; primarily through the use of its non-enzyme electrode. However, such systems are limited because the system gives little indication of the exact nature of the fluidics issue involved in the failure. Since failed blood samples set off alarms and prevent the system from reporting blood glucose data, a fluidics malfunction in the in-dwelling portion of the disposable (which is one of the most common causes of blood sampling failures) is very troublesome for the user. Current systems do not attempt to correct fluidics issues before alerting the user, and when it does alert the user of a blood sampling failure, it gives a very general indication of failure. This often causes clinician frustration and creates an inefficient fluidics debugging process.

### SUMMARY

In the embodiments of the present disclosure, a method for analyzing failed bodily fluid sampling is provided. The method includes: providing an analyte sensing system having a sensor coupled to a sampling line and an access device configured to sample bodily fluid, the access device having an inner space, wherein the sampling line is positioned in the inner space, the analyte sensing system comprising a flow controller configured to draw bodily fluid through the sampling line and inner space of the access device and flush a fluid through the sampling line and inner space and a monitor in communication with the sensor and the flow controller, the monitor comprising a computer apparatus including a processor and a memory; performing, via the sensing system, a sampling event of a subject's vasculature; obtaining, continuously and/or intermittently, one or more status values of the sampling event via the sensing system; comparing, via the sensing system, the one or more status values with at least one predetermined value corresponding to a failed sampling event; and determining, via the sensing system, whether the sampling event failed based on the comparing step, wherein in the event of the sampling event failing, the method further comprising: flushing or drawing the sampling line or a portion of the inner space of the access device via the flow controller.

In some embodiments of the method, the method further includes: detecting pressure in response to flushing or drawing the sampling line or the portion of the inner space; and determining one or more possible root causes for the failure of the sampling event based on the detected pressure. In other embodiments, the method includes: in response to determining the one or more possible root causes, terminating the sensing system, requesting user intervention, performing flushing of the sampling line, or performing flushing of the portion of the inner space. In still other embodiments, the method includes: detecting a normal pressure in response to flushing the sampling line or the portion of the inner space of the access device; detecting an increased pressure in response to drawing the sampling line or the portion of the inner space of the access device; determining that the failed sampling event is caused by a port of the access device being in contact with the wall of the subject's vasculature containing the access device. In further embodiments, the method further includes stop the analyte sensing system; and request the user to adjust the position of the access device.

In further embodiments of the method, the method further includes detecting an increased pressure in response to flushing or drawing the sampling line or the portion of the inner space of the access device; detecting, in response to flushing the sampling line, high compliance; determining that the failed sampling event is caused by the access device kinking at a location that is distal to the distal end of the sampling line. In other embodiments, the method includes detecting an increased pressure in response to flushing or drawing the sampling line or the portion of the inner space of the access device; detecting, in response to flushing the sampling line, low compliance; determining that the failed sampling event is caused by the kinking of the access device and the sampling line, the access device kinking at a location that is proximal to the distal end of the sampling line.

In still further embodiments of the method, the method includes detecting a normal pressure in response to flushing or drawing the portion of the inner space of the access device; detecting an increased pressure in response to flushing or drawing the sampling line; determining that the failed sampling event is caused by a clot formed in the internal lumen of the sampling line. In other embodiments, the method includes performing a high pressure flush of the sampling line to dislodge the clot. In some embodiments, the method includes detecting a normal pressure in response to flushing or drawing the portion of the inner space of the access device; detecting a normal pressure in response to flushing the sampling line; detecting an increased pressure in response to drawing the sampling line; determining that the failed sampling event is caused by a clot formed on the tip of the sampling line. In still other embodiments, the method includes performing, simultaneously, a high pressure flush of the sampling line and the portion of the inner space of the access device to dislodge the clot.

In further embodiments of the present disclosure, a method for analyzing failed bodily fluid sampling is provided. The method includes: providing an analyte sensing system having a sensor coupled to a sampling line and an access device configured to sample bodily fluid, the access device having an inner space, wherein the sampling line is positioned in the inner space, the analyte sensing system comprising a flow controller configured to draw bodily fluid through the sampling line and inner space of the access device and flush a fluid through the sampling line and inner space and a monitor in communication with the sensor and the flow controller, the monitor comprising a computer apparatus including a processor and a memory; performing, via the sensing system, a sampling event of a subject's vasculature; obtaining, continuously and/or intermittently, one or more status values of the sampling event via the sensing system; comparing, via the sensing system, the one or more status values with at least one predetermined value corresponding to a failed sampling event; and determining, via the sensing system, whether the sampling event failed based on the comparing step, wherein in the event of the sampling event failing, the method further comprising: flushing or drawing the sampling line or a portion of the inner space of the access device via the flow controller; detecting pressure in response to flushing or drawing the sampling line or the portion of the inner space; and determining one or more possible root causes for the failure of the sampling event based on the detected pressure.

In other embodiments of the method, the one or more possible root causes for the failure of the sampling event comprises at least one of (i) a port of the access device contacts the wall of the subject's vasculature containing the access device; (ii) the access device kinks at a location that is distal to the distal end of the sampling line; (iii) the access device kinks at a location that is proximal to the distal end of the sampling line; (iv) a clot forms in the internal lumen of the sampling line; and (iv) a hanging clot forms on the tip of the sampling line. In still other embodiments, the method includes, in response to determining the one or more possible root causes, terminating the sensing system, requesting user intervention, performing flushing of the sampling line, or performing flushing of the portion of the inner space.

In some embodiments of the method, the one or more possible root causes for the failure of the sampling event comprises at least one of (i) a port of the access device contacts the wall of the subject's vasculature containing the access device; (ii) the access device kinks at a location that is distal to the distal end of the sampling line; (iii) the access device kinks at a location that is proximal to the distal end of the sampling line; (iv) a clot forms in the internal lumen of the sampling line; and (iv) a hanging clot forms on the tip of the sampling line. In other embodiments of the method, in the event of the sampling event failing, the method further includes: drawing bodily fluid through the sample line or a portion of the inner space of the access device; detecting pressure in response to drawing bodily fluid through the sample line or a portion of the inner space of the access device; and determining one or more possible root causes for the failure of the sampling event based on the detected pressure. In still other embodiments, in response to determining the one or more possible root causes, terminating the sensing system, requesting user intervention, performing flushing of the sampling line, or performing flushing of the portion of the inner space.

Also provided herein is a system for analyzing failed bodily fluid sampling. The system includes: a sensor coupled to a sampling line and an access device configured to sample bodily fluid, the access device having an inner space, wherein the sampling line is positioned in the inner space; a flow control system configured to draw bodily fluid through the sampling line and inner space of the access device and flush a fluid through the sampling line and inner space; and a monitor in communication with the sensor and flow control system, the monitor comprising a computer apparatus including a processor and a memory; and a bodily fluid sample analysis module stored in the memory, comprising executable instructions that when executed by the processor cause the processor to: obtain, continuously and/or intermittently, one or more status values of the sampling event; compare the one or more status values with at least one predetermined value corresponding to a failed sampling event; and determine whether the sampling event failed based on the comparing step; cause the flow control system to flush or draw the sampling line or a portion of the inner space of the access device.

In some embodiments of the system the module is further configured to: cause the sensor to detect pressure in response to flushing or drawing the portion of the inner space of the access device or the sampling line; and determine one or more possible root causes for the failure of the sample based on the detected pressure. In other embodiments, in response to determining the one or more possible root causes, terminate the sensing system, request user intervention, perform flushing of the sampling line, or perform flushing of the portion of the inner space. In still other embodiments, the module is further configured to: detect a normal pressure in response to flushing the sampling line or the portion of the inner space of the access device; detect an increased pressure in response to drawing the sampling line or the portion of the inner space of the access device; determine that the failed sampling event is caused by a port of the access device being in contact with the wall of the subject's vasculature containing the access device.

In further embodiments of the system, the module is further configured to: detect an increased pressure in response to flushing or drawing the sampling line or the portion of the inner space of the access device; detect, in response to flushing the sampling line, high compliance; determine that the failed sampling event is caused by the access device kinking at a location that is distal to the distal end of the sampling line. In other embodiments, the module is further configured to: detect an increased pressure in response to flushing or drawing the sampling line or the portion of the inner space of the access device; detect, in response to flushing the sampling line, low compliance; determine that the failed sampling event is caused by the kinking of the access device and the sampling line, the access device kinking at a location that is proximal to the distal end of the sampling line.

In still further embodiments of the system, the module is further configured to: detect a normal pressure in response to flushing or drawing the portion of the inner space of the access device; detect an increased pressure in response to flushing or drawing the sampling line; determine that the failed sampling event is caused by a clot formed in the internal lumen of the sampling line. In other embodiments, the module is further configured to: detect a normal pressure in response to flushing or drawing the portion of the inner space of the access device; detect a normal pressure in response to flushing the sampling line; detect an increased pressure in response to drawing the sampling line; and determine that the failed sampling event is caused by a clot formed on the tip of the sampling line.

These and other features and advantages of the present disclosure will become more readily apparent to those skilled in the art upon consideration of the following detailed description and accompanying drawings, which describe both the preferred and alternative embodiments of the present disclosure.

### BRIEF DESCRIPTION OF THE DRAWINGS

FIG. 1 is a perspective view of an analyte sensing system of one embodiment of the present disclosure;

FIG. 2 is a side view of an access device assembly in accordance with various embodiments of the present disclosure;

FIG. 3 is a perspective view of a catheter in accordance with various embodiments of the present disclosure;

FIG. 4 is a schematic of a rotary pinch valve of a flow control system in accordance with various embodiments of the present disclosure;

FIG. 5A is a schematic of a flow module system in accordance with various embodiments of the present disclosure;

FIG. 5B is a schematic of a flow module system in accordance with various embodiments of the present disclosure;

FIG. 6 is an enlarged view of an access device and sampling line in accordance with various embodiments of the present disclosure;

FIG. 7 is an enlarged view of an access device and sampling line in accordance with various embodiments of the present disclosure;

FIG. 8 is an enlarged view of a sampling line in accordance with various embodiments of the present disclosure;

FIG. 9 is a flowchart of a system and method for analyzing and correcting blood sampling failure in accordance with various embodiments of the present disclosure;

FIG. 10 is a block diagram of a system and environment for analyzing and correcting blood sampling failure in accordance with various embodiments of the present disclosure; and

FIG. 11 is a graphical depiction of a flow profile of an embodiment of the present disclosure.

### DETAILED DESCRIPTION

The present disclosure now will be described more fully hereinafter with reference to specific embodiments of the present disclosure. Indeed, the present disclosure can be embodied in many different forms and should not be construed as limited to the embodiments set forth herein; rather, these embodiments are provided so that this disclosure will satisfy applicable legal requirements. As used in the specification, and in the appended claims, the singular forms “a”, “an”, “the”, include plural referents unless the context clearly dictates otherwise. The term “comprising” and variations thereof as used herein is used synonymously with the term “including” and variations thereof and are open, non-limiting terms.

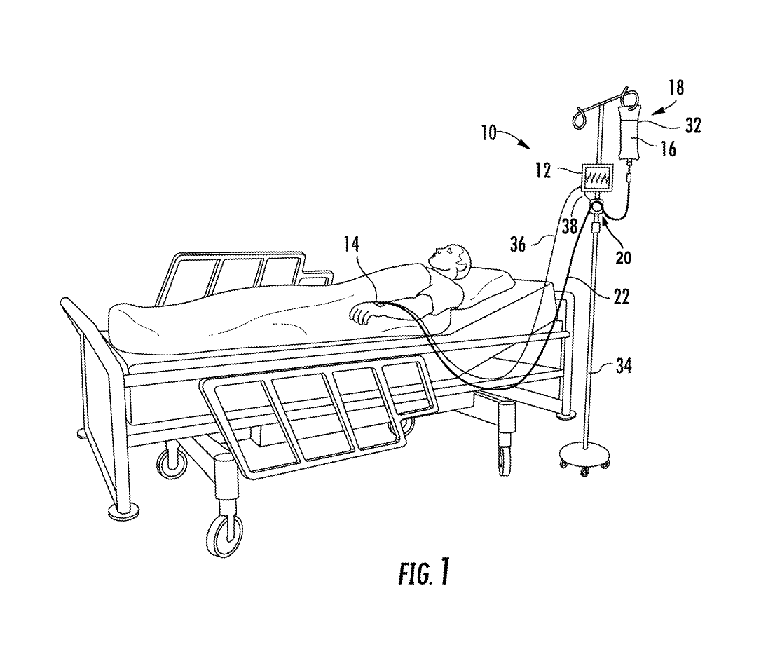

Embodiments of the present disclosure include a blood glucose sensing system 10 that includes a monitor 12, a sensor assembly 14, a calibration solution source 16 and a flow control system 18, as shown in FIG. 1. Notably, the present disclosure could also be employed with other analyte or blood parameter sensing systems that require drawing of blood or fluid samples from a patient. Blood, as used herein, should be construed broadly to include any body fluid with a tendency to occlude lumens of various body-access devices during sampling. The flow control system 18 includes a flow controller 20, a monitor line 22, a sensor casing (not shown), an adapter (not shown), a sampling line 228 and a catheter 30, as shown in FIGS. 1, 2, and 3. Generally, the flow control system 18 of one embodiment of the present disclosure is configured to mediate flow of small volumes of the calibration solution 16 over the sensor assembly 14 and withdraw small volumes of samples of the blood from the patient for testing by the sensor assembly.

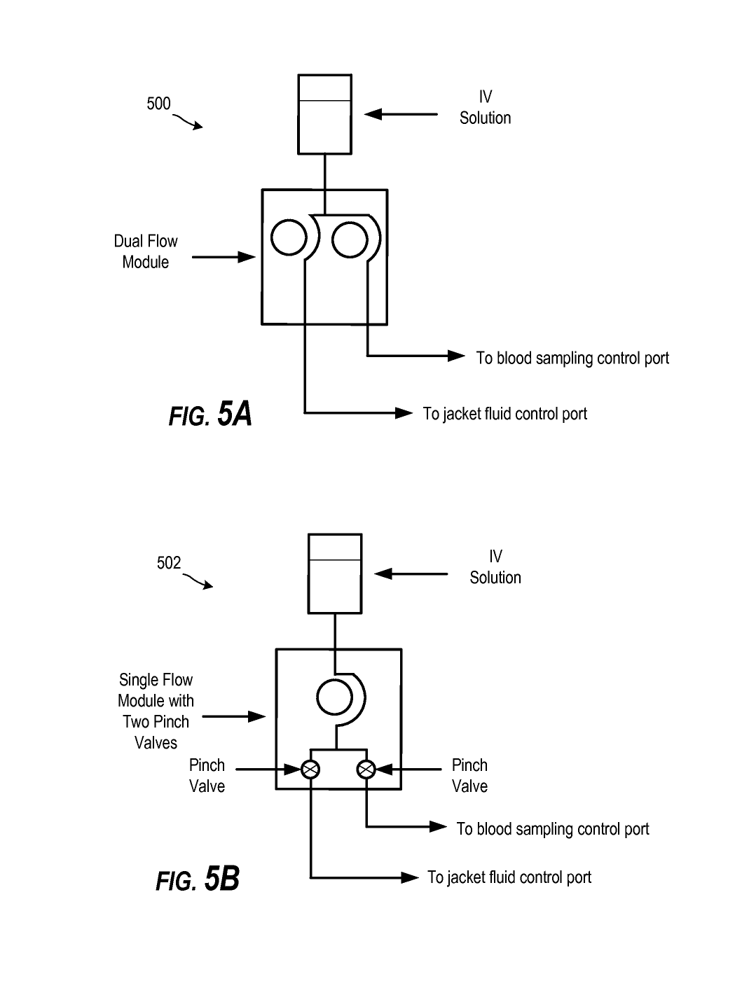

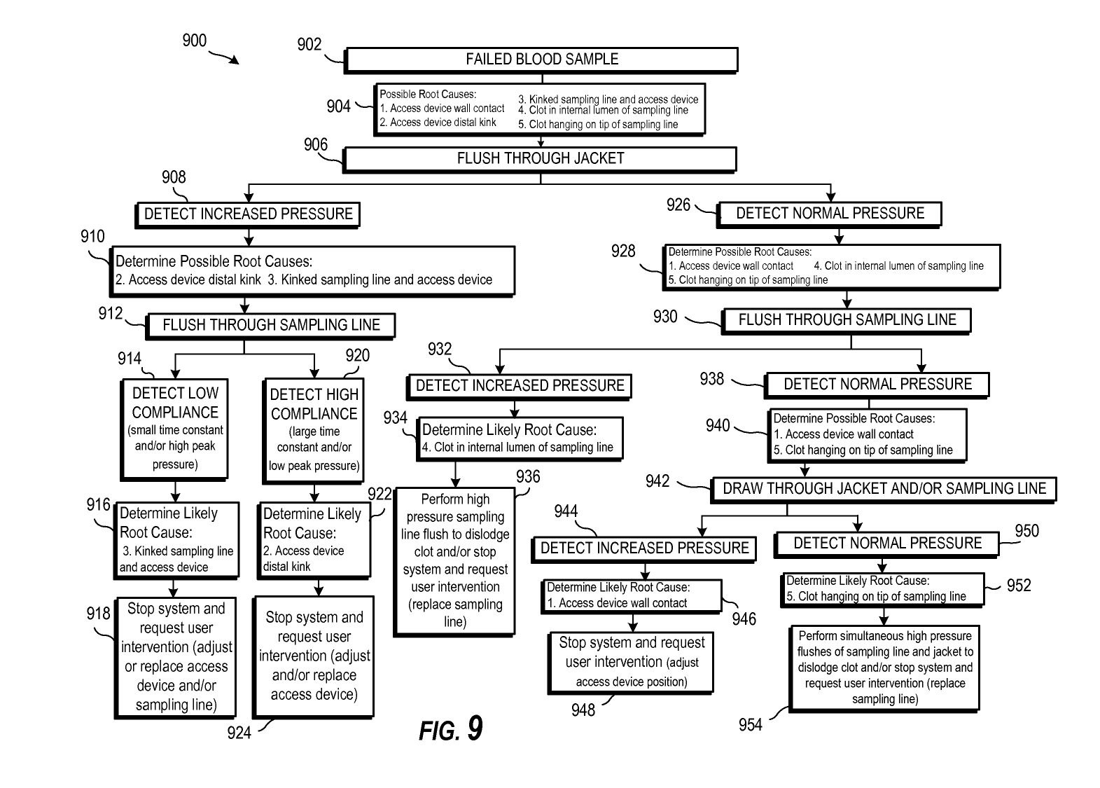

The flow control system 18 in another embodiment is able to support the flush and draw pressures and volumes, and the high number of sampling cycles over a long multi-day indwell, needed for continuous analyte (glucose) monitoring, while avoiding the formation of thrombi that occur in conventional catheters by providing a small-diameter, smooth and relatively void free surface defining a lumen extending up to the sensor assembly 14. In some embodiments, the flow control system 18 includes the flow module system 500 of FIG. 5A or the flow module system of FIG. 5B. In another embodiment, the sampling line 228 of the flow control system 18 may be employed with a range of existing catheter 30 configurations by having the sampling line sized and configured for insertion into a lumen of an existing catheter. In still other embodiments of the present disclosure, thrombus formation is inhibited by balancing the structure of various components of the flow control system 18 and operation of the flush and draw cycles by the flow controller 20 as explained in more detail below with regard to FIG. 9.

The monitor 12 is connected in communication with the sensor assembly 14 through communication lines or wires 36 and to the flow control system 18 through communication lines or wires 38, as shown in FIG. 1. These communication lines 36, 38 could also represent wireless data communication such as cellular, RF, infrared or blue-tooth communication. Regardless, the monitor 12 includes some combination of hardware, software and/or firmware configured to record and display data reported by the sensor assembly 14. For example, the monitor may include processing and electronic storage for tracking and reporting blood glucose levels. In addition, the monitor 12 may be configured for automated control of various operations of other aspects of the sensing system 10. For example, the monitor 12 may be configured to operate the flow control system 18 to flush the sensor assembly 14 with calibration solution from source 16 and/or to draw samples of blood for testing by the sensor assembly. Also, the monitor 12 can be configured to calibrate the sensor assembly 14 based on the flush cycle.

The sensor assembly 14 includes a wire electrode sensor (e.g., the sensor 204) that includes, for example, a glucose-oxidase coated platinum wire covered by a membrane that selectively allows permeation of glucose. The glucose-oxidase responds to the glucose by generating hydrogen peroxide which, in turn, generates an electrical signal in the platinum wire. The wire electrode sensor may include some processing component and/or just communicate the signal up through the communication wires 36 attached thereto for further processing by the monitor 12. The sensor assembly 14 may also include counter and/or reference wire electrodes bundled with the working electrode. Regardless, in the illustrated embodiment, the wire electrode sensor is adapted to be within the flow path of the blood sample, as will be described in more detail herein below.

It should be noted that, although particularly advantageous for sensors directly within the flow path of the blood sample, the particular configuration of the sensor assembly 14 that puts it within the flow of the blood and/or calibrant path may vary and still be within the scope of the present disclosure. For example, the sensor could be a microfluidics sensor that is adjacent to, and routed off of, a portion of the flow control system 18 within the reach of a blood volume draw. Also, the sensor could be an optical or vibrational sensor that senses blood parameters without contact with the blood sample, such as through a vibrationally or optically transparent adjacent portion of the flow control system.

The calibrant solution source 16 is supplied, in one embodiment, from a bag 32 mounted on a pole 34. The calibrant solution supply is preferably off-the-shelf and/or not inconvenient to employ in a hospital setting and is also beneficial to the patient and includes attributes that help with function of the glucose sensing system 10. For example, the solution in the bag may be a Plasmalyte or conventional saline with selected amounts of buffers and anti-thrombogenic compounds, such as heparin, that help with flushing the sensor assembly 14 to keep it clear of clots and thrombosis. The solution in the bag 32 may also include various nutrients to keep fluid and nutrition at appropriate levels for the patient. Although the illustrated embodiment employs a fluid bag 32, it should be noted that the calibrant solution source 16 could include several sources, including several sources at one time, and have varying compositions. For example, a pressurized canister or a reservoir may be employed.

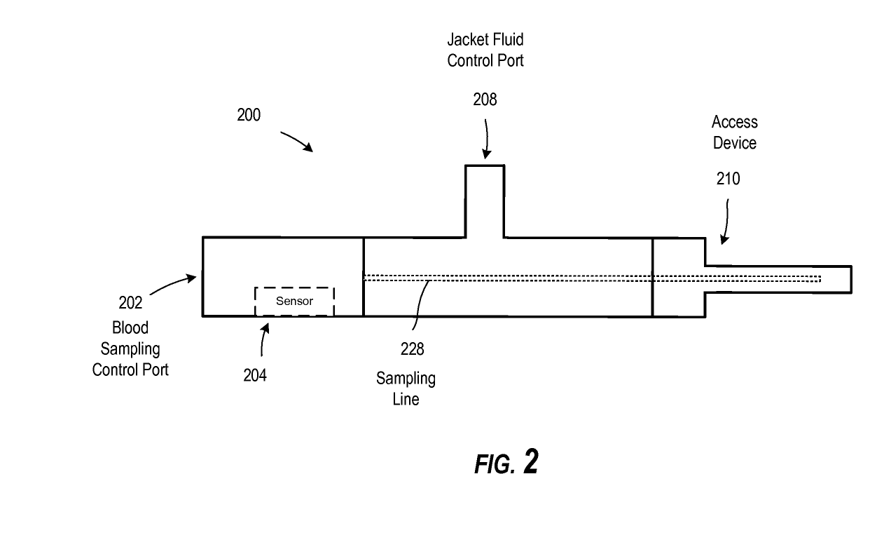

Referring now to FIG. 2, an exemplary access device assembly 200 is provided. The access device assembly 200 includes a blood sampling control port 202 for receiving the sampling line 228, a sensor 204, a jacket fluid control port 208, and an access device 210. The access device, in some embodiments, includes the catheter 30. The sampling line 228, in some embodiments, is a small tube that is placed inside of the access device being used. As used herein, “jacket” or “jacket space” refers to the internal volume of the access device apart from the space being filled by the sampling line. As shown in the illustrated embodiment, a “t” junction or similar device is employed so that the jacket space can be controlled separately from the sampling line.

As shown in FIG. 1, the monitor line 22 of an embodiment of the flow control system 18 extends from the calibrant solution source 16 through the flow controller 20 and attaches to the rest of the flow control system 18 (sensor casing, adapter and sampling line 228 within catheter 30) closer to the sensor assembly 14. Preferably, the monitoring line is an 8 foot length of PVC extension tubing with a 0.0625 inch internal diameter.

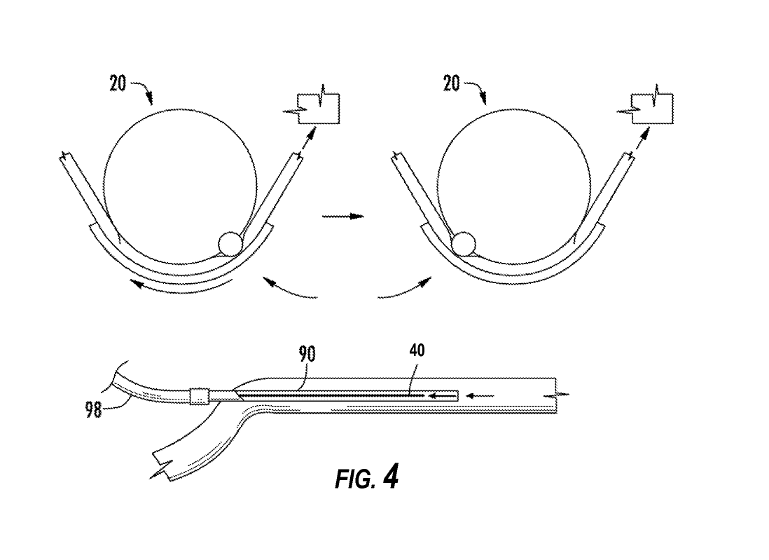

The flow controller 20 in one embodiment of the present disclosure includes some type of hardware, software, firmware or combination thereof that electromechanically controls one or more valves, or other mechanical flow control devices, to selectively allow or stop flow through the monitor line 22. In the illustrated embodiment of FIG. 4, the mechanical aspect of the flow controller 20 includes a rotary pinch valve through which extends the monitor line 22. This rotary pinch valve pinches the fluid line to stop flow and, by sliding along a short length of the fluid line, can advance or retract the calibrant solution or retract the calibrant solution supply in a column extending down to the end of the catheter 30. Different numbers of roller heads may be used, such as two or four heads, the latter aiding with higher draw volumes.

Notably, the flow controller 20 of the illustrated embodiment employs a combination of the head (primarily, except for the short draw and infusion by pinch point advancement) generated by the elevation of the fluid bag 32 on the pole 34 and the on-off regulation of the flow induced by the head. One advantage, however, of the illustrated embodiment is that the gravity feed of the fluid bag 32 on the pole 34 is well-understood and mediated to control the amount of fluid administered to the patient. Regardless, the role of the flow controller 20 can be met flexibly with various combinations of technology and the present disclosure shouldn't necessarily considered limited to any one particular configuration.

When the flow controller 20 opens its pinch valve, solution from the bag 32 is gravity fed down through the monitor line 22, the sensor casing, the adapter, the sampling line 228 and (if used) the catheter 30 and into the patient's vasculature. Or, the flow controller 20 could advance the pinch valve in the direction of the catheter 30 and drive the solution to flush the sensor 40 and out through the catheter. If the solution from the bag 32 includes heparin or other anti-thrombogenic agent and/or some anti-thrombogenic mechanical qualities, this flush step clears the catheter and cleans the sensor 40.

In a draw step, the pinch valve is reversed by the flow controller 14 forming a vacuum and drawing a blood sample up into the catheter from the patient's vasculature. The glucose sensor, during or after this step, can then be activated to sense the glucose concentration in the blood sample. After sufficient time has elapsed to take one or more analyte measurements, the flush cycle is then run, typically in 5 to 10 minute cycles, as described above. This process of flush-and-draw is repeated over the life of the sensing system 10, or at least the life of the glucose sensor. The description above is a more general overview of the flush/draw process. Variations in the specifics of the flush and draw cycles and how they're adapted to work with the present system to avoid thrombosis, minimize flush and draw volumes and work with existing catheter configurations will be described in more detail below.

In an embodiment of the present disclosure, the flow profile preferably lasts for 5 to 7.5 minutes and delivers less than 500 mL of solution from the bag 32 over a 72-hour period. Also, the flow controller 14 preferably has improvements to ensure accuracy and repeatability of its control of fluid flow through the flow control system 18. For example, the above-described rollers may be accompanied by an encoder coupled with a stepper motor that provides redundant control of the roller head orientation. Also, there may be an air detection sensor distal to the roller head assembly that uses optical or ultrasonic sensing (an ultrasonic pulse) to detect gas or liquid conditions in the tube segment.

FIGS. 5A-5B are exemplary embodiments of flow module systems. In some embodiments, a flow module system 500 and a flow module system 502 include the flow controller 20. As illustrated in FIG. 5A, the flow module system 500 includes a dual-flow module, where IV solution (e.g., the calibrant 16) is flushed into the access device assembly 200 through the blood sampling control port 202 and also through the jacket fluid control port 208. In FIG. 5B, the flow module system 502 includes a single flow module with two pinch valves such that the IV solution can be flushed into the access device assembly 200 through the blood sampling control port 202 and the jacket fluid control port 208. The flow module system 500 and the flow module system 502 also draw blood from the blood pathway to the sampling line and/or jacket space. In this way, the flow module system 500 and the flow module system 502 independently controls the two different fluid pathways, i.e., the sample line and the jacket fluid.

In some embodiments, the sampling line 228 is used in combination with a sampling tube 90 (see, FIG. 4). The sampling tube 90, in one embodiment, is a very small ID tube that has a relatively large OD and is constructed of a material that's mechanically thromboresistant (and may be combined with heparin or other anti-thrombosis agents) due to its internal shape, smoothness and void-free structure. Without being wed to theory, it is believed that the smaller ID is less prone to clotting or other thrombosis since the pressure profile across the cross-section of the blood is more evenly distributed because the red blood cells and other blood components are a larger percentage of the cross section of the lumen defined therethrough. More even pressure distribution helps to ensure that the blood components do not stop against the side of the lumen walls of the sampling tube 90, cutting down on the tendency to clot. In addition, the smaller ID reduces the size of the flush and draw amounts to minimize side effects on the patient. Less blood in the draw means lower flushing volumes with the heparin in the calibration solution.

The relatively larger OD of the sampling tube 90 is advantageous in that it provides a good buckling stiffness to enable insertion of the sampling tube 90 directly into the patient (preferably in combination with a needle or other introducer) or into the lumen of an existing catheter 30 without bending or kinking. Still, if such a combination is desired, the OD can be constrained to allow the sampling line 228 to be combined with existing catheters or introducers. In one embodiment, for example, the sampling line has an outer diameter of 0.030 inch configured to fit within a range of standard-sized catheter 30 lumens, such as the three-lumen MULTI-MED central venous catheter or an ADVANCED VENOUS ACCESS (AVA) catheter (Edwards Lifesciences, Irvine, Calif.). Despite the aforementioned preferred configurations and sizes, a balance may be struck between a range factors, flow rates, adaptability to existing catheters, anti-thrombotic attributes and the ID/OD, length and other attributes of the sampling tube 90 to create other embodiments of the present disclosure as will be described more below.

The advantage of inserting the sampling tube 90 into an existing catheter 30 is that a dedicated line for sampling the analyte or blood parameter is no longer needed. In addition, the sampling tube 90 can reduce the cross-sectional area through which blood is drawn to reduce clotting and sample volume. Further, the sampling tube 90 can serve as a sleeve that covers the gaps, transitions and other voids that are present in conventional catheters.

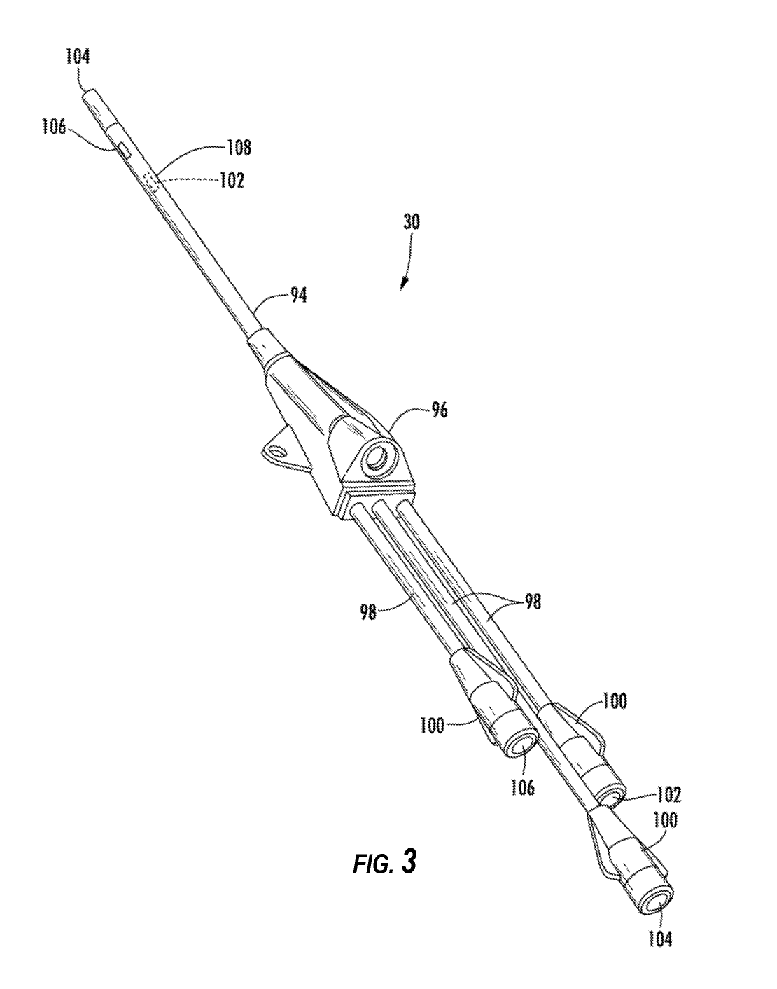

Conventional catheters 30, for example the catheter shown in FIG. 3, frequently include three parts, a multi-lumen tube 94, a back form 96 and lines 98. The multi-lumen tube 94 inserts into the patient and provides lumens that exit at different points of the multi-lumen tube depending upon the function employed with each lumen. For example, one lumen may be a supply lumen 102 for administering drugs that exits at the distal end of the tube 94, another sensing lumen 104 for communicating with a pressure sensor for determining cardiac output that exits at a midpoint from the side of the tube 94 and a third sampling lumen 106 for sampling blood that exits at a proximal point 108 from the side of the tube 94.

Each of the lumens within the multi-lumen tube 94 communicates with a dedicated channel defined in the back form. These channels diverge within the back form 96 (which typically has a triangular shape as it extends away from the patient) and each of the channels connects up with a dedicated one of the lines 98. Each time a transition between the components 94, 96, 98 occurs, there are discontinuities, gaps, rough surfaces, material variations and other voids that might promote the occurrence of clotting and other thrombosis and/or require less-desirable flow rates for the long-term, high-count sampling needed for the present disclosure.

In one embodiment of the present disclosure, the sampling line 228 connects, via a locking cap (not shown), to a luer lock 100 mounted on the proximal end of one of the lines 98 that communicates through the back form 96 with the sampling lumen 106 of the catheter 30. The sampling tube 90 extends through the line 98 and the back form 96 and partially through the sampling lumen 106, stopping about 1 inch short of the proximal exit point 108. Advantageously, the proximal exit point avoids draw of blood samples diluted or otherwise affected by the operations being performed in the other lumens 102, 104. Also, the sampling tube 90 provides a void-free lumen that bypasses the voids formed by the junctions between the components 94, 96, 98, and the varied internal contours of those components, so as to reduce clotting and the volume of blood draws needed to supply the sensor 40. Stopping short of the proximal exit port 108 avoids extension of the sampling tube 90 out of the exit port and making contact with the patient's vasculature.

As another alternative, the sampling tube 90 may be of sufficient length to extend out of the exit port 108. This embodiment has the advantage of extending the void-free internal diameter of the sampling tube 90 past any irregularities at the end of the sampling lumen 106.

The length of the sampling tube 90 can be selected based on a range of factors. In the embodiment described above, the sampling tube 90 is configured to end about an inch short of the proximal exit port 108. This is because the variations in length of conventional catheters within a model can be relatively high (+/−1 inch) from the back form 96 through the extension lines 98. Longer length sampling tube 90s would be required for peripherally inserted central catheters (PICC), and could be 40 or even 60 cm long. Alternatively, the sampling tube 90 could be much shorter and only extend past those regions of the catheter 30 with thrombosis generating qualities, such as past the junction between the back form 96 and the tube 94 or whichever catheter regions are expected to be most prone to thrombus formation. For example, the CVC catheter may be 13.4 inches long but the sampling tube 90 only 1.97 inches long. Shorter sampling tube 90s, however, are expected to use a two-stage blood draw process wherein the blood is first drawn into the catheter 30 and then later drawn into the sampling line 228.

The length of sampling line 228 (and adapter) could be selected on the proximal end to ensure a protective guard for the sensor. Also, the length of the sampling tube 90 could be selected for ensuring sufficient durability of the combined sampling line 228 and catheter 30, or could be selected to provide sufficient area for application of an anti-clotting coating. Lengths could also be varied to fit standard catheter 30 model lengths, allowing a healthcare worker to select and couple the catheter with the sampling line 228 at the time of insertion. Lengths can range for CVC's from 16 inches, 20 inches and 30 inches, for example. Other lengths are also possible for different types of access devices, such as PICC's and IV catheters and introducers.

In one embodiment, the sampling tube 90 has a constant 0.010 inch ID and a 0.025 inch OD so as to fit a range of standard-sized catheters 30. Also, the OD might be even smaller, such as 0.15 inch with a 0.010 inch ID, but the ID may be scaled down to keep bending stiffness high, such as down to 0.008 inch. The dimensions of the sampling tube 90 and sampling line 228 need not be consistent through its entire length.

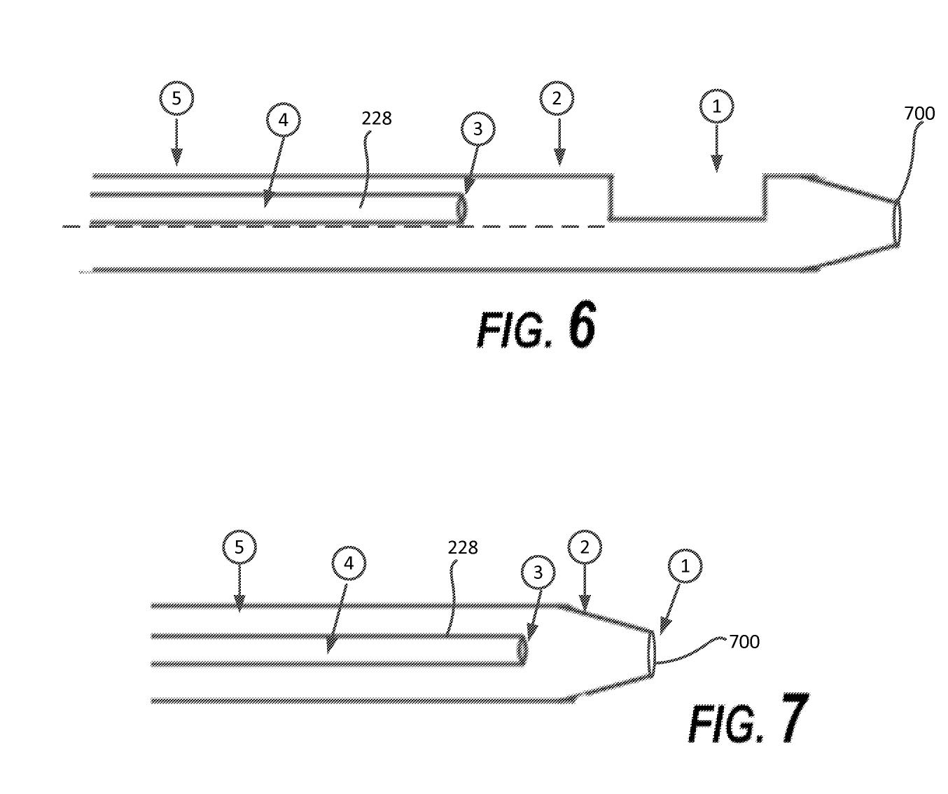

FIGS. 6 and 7 will now be discussed to demonstrate the locations of at least five root causes for failed blood samples. In FIG. 6, the sampling line 228 placed within the inner space of access device 600 is illustrated. In the illustrated embodiment, the sampling line is placed with the proximal lumen of a Central Venous Catheter. FIG. 7 illustrates the sampling line 228 placed within the inner space of access device 700, where the access device 700 comprises a peripheral intravenous catheter.

Five exemplary root causes for failed blood sampling are set forth in Table 1 below.

TABLE 1

| | Flush Through | Draw Through | Flush Through |
|---|---|---|---|
| Root Cause | Sampling Line | Jacket | Jacket |
| Access device wall | Normal | Increased | Normal |
| contact | Pressure | Pressure | Pressure |
| Access Device | Increased | Increased | Increased |
| Distal Kink | pressure (high | pressure | pressure |
| | compliance) | | |
| Kinked sampling line | Increased | Increased | Increased |
| and access device | pressure (low | pressure | pressure |
| | compliance) | | |
| Clot in internal lumen | Increased | Normal | Normal |
| of sampling line | pressure | pressure | pressure |
| Clot hanging on tip | Normal | Normal | Normal |
| of sampling line | pressure | pressure | pressure |

The five exemplary root causes for failed blood sampling are further explained as follows.

1. The port of the access device contacts the wall of the vessel containing the access device (see, FIGS. 6 and 7, locations 1). In this case, an attempt to draw fluid through either the sampling line or the jacket will cause the vessel wall to occlude the port of the access device, causing increased pressure readings. Flushing either the sampling line or the jacket should display normal pressure readings. The vessel can be in vivo or external to a human or animal. For example, the vessel may include veins, arteries, or an external container containing fluid to be sampled.

2. The access device kinks at a location that is distal to the distal end of the sampling line (see, FIGS. 6 and 7, locations 2). For example, the access device may bend, twist, or otherwise deform at locations 2. In this case, drawing or flushing through the sampling line or the jacket should display increased pressures. However, since fluid flushed through the sampling line can exit the sampling line and enter the jacket of the access device, the full compliance (ΔV/ΔP) observed during a flush of the sampling line should be relatively high. Depending on the severity of the occlusion, this will cause a relatively long time-constant or a relatively gradual slope in the pressure waveform and/or a relatively low final pressure magnitude.

3. The access device kinks at a location that is proximal to the distal end of the sampling line, thus kinking the sampling line as well (see, FIGS. 6 and 7, locations 5). In this case, as in case 2, drawing or flushing through the sampling line or the jacket should display increased pressures. But in this case, since fluid flushed through the sampling line cannot exit the sampling line, the full compliance observed during a flush of the sampling line should be relatively low. Depending on the severity of the occlusion, this will cause a relatively short time-constant or a relatively steep slope in the pressure waveform and/or a relatively high final pressure magnitude.

4. A clot forms in the internal lumen of the sampling line (see, FIGS. 6 and 7, locations 4). In this case, drawing or flushing through the sampling line will display increased pressures, while drawing or flushing through the jacket of the access device will be unaffected.

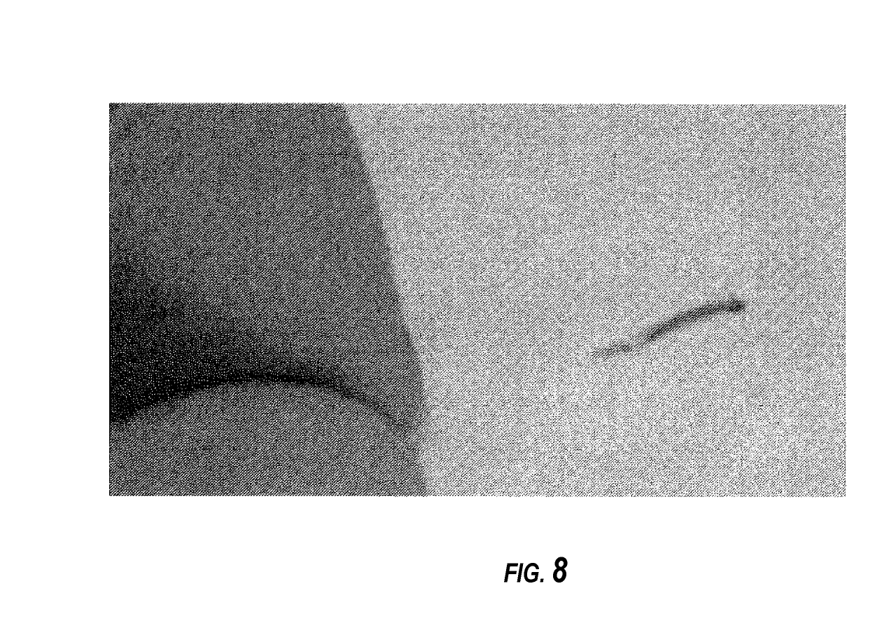

5. A hanging clot forms on the tip of the sampling line (see, FIGS. 6 and 7, locations 3). If a hanging clot forms on the tip of the sampling line, drawing or flushing through the access device jacket will be unaffected. Flushing through the sampling line will also be unaffected, as flushing will cause the free-moving part of the hanging clot to move away from the distal tip of the sampling line. However, drawing through the sampling line will display increased pressures, as drawing through the sampling line causes the free-moving part of the hanging clot to move toward and onto the distal tip of the sampling line, thus occluding it. FIG. 8 is a picture taken of a representative clot observed in a 72-hour sheep study that caused this phenomenon.

In order to analyze (and attempt to correct) any blood sampling failures, the system must have the ability to differentiate between the possible root causes of a given sampling failure. One way to accomplish this is by performing a series of draws and flushes through the sampling line and “jacket” and observing the consequent pressures, as outlined in detail above. Using the pressure characteristics of these draws and flushes allows the system to arrive at a most likely root cause for the blood sampling failure. After the root cause is identified, the system can either automatically take the appropriate action to attempt to correct the problem if possible, or it can prompt the user with a specific root cause to investigate.

An advantage of this automated blood sampling failure analysis and correction technique is user convenience. If the glucose monitoring system produces frequent alerts of failed blood draws but no automatic correction or root cause analysis, the burden to debug the system is placed wholly on the clinician. Using an algorithm, the system can reduce the frequency of user intervention (by correcting some sampling issues automatically) and can improve the efficiency of any user intervention that is still required (by directing the clinician to the root cause of the sampling failure)

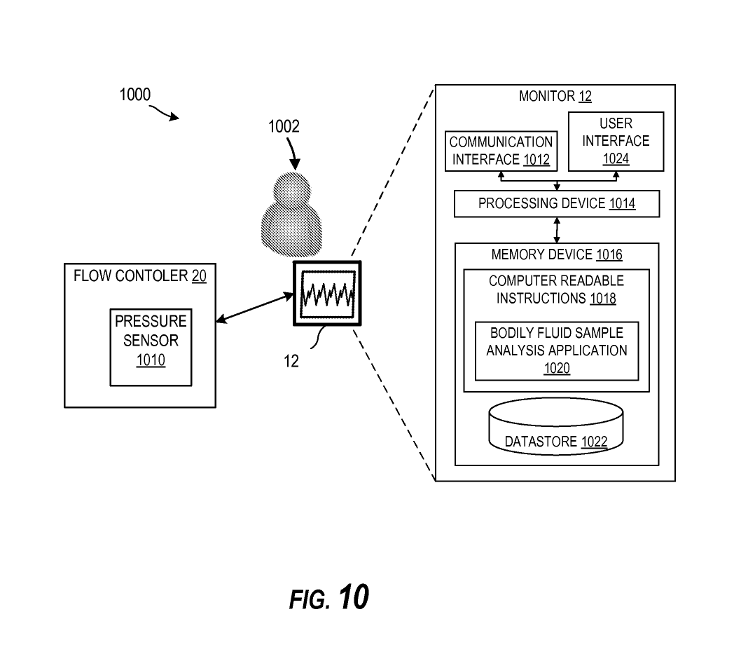

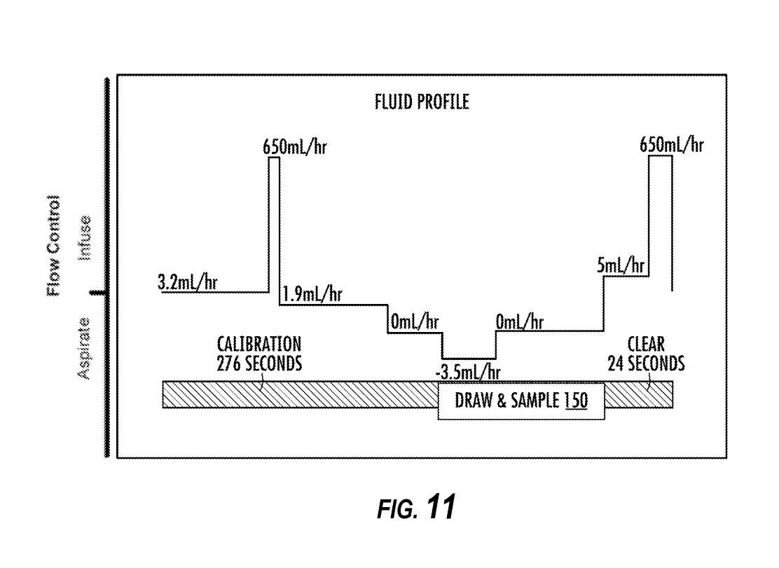

Referring now to FIG. 9, a process flow 900 of analyzing and correcting a failed blood sample is illustrated. In some embodiments, the process flow 900 is performed by an apparatus having hardware and/or software configured to perform one or more portions of the process flow 900 (e.g., the pressure sensor 1010, the fluid controller 20, and/or the monitor 12 of FIG. 10).

As illustrated at block 902, a failed blood sample is provided. Possible root causes for the failed blood sample include: 1) Access device wall contact; 2) Access device distal kink; 3) Kinked sampling line and access device; 4) Clot in internal lumen of sampling line; and 5) Clot hanging on tip of sampling line as illustrated at block 904. In some embodiments, a determination that the blood sample has failed is based on a reading provided on the display of monitor 12. For example, the display of monitor 12 may provide an error message. Failed blood samples include, for example, samples where little or no blood is drawn, samples in which the blood is defective (e.g., dilute with calibrant), samples attributable to signal noise, and the like. Upon obtaining a blood sample from the subject's vessel, one or more status values of the blood are obtained continuously or intermittently. In exemplary embodiments, the one or more status values is compared with at least one predetermined value corresponding to a failed blood sample and it is determined that the blood sample has failed based on the comparison.

In response to determining that the blood sample has failed, the jacket of the access device is flushed as illustrated at block 906. For example, the flow module system 500 or 502 may be used to flush the jacket with IV solution. In response to flushing through the jacket, an increased pressure or a normal pressure is detected, as illustrated at blocks 908 and 926, respectively. In some embodiments, pressure is detected by a pressure device (e.g., the pressure sensor 1010 of FIG. 10) that is associated with the flow module system 500 and/or the flow module system 502. The pressure device may be, for example, incorporated into the flow controller 20 or be external thereto, e.g., interfaced with tubing. Exemplary pressure devices include Edwards TruWave Disposable Pressure Transducer from Edwards Lifesciences, LLC. The pressure device detects the pressure of fluids in a line or tubing leading out of the blood sampling control port 202 or the jacket fluid control port 208, and converts the pressure to a signal that can be communicated to the monitor 12. Pressure can be sensed directly (i.e., via direct physical contact with fluid such as blood) or indirectly (e.g., optically).

In cases where an increase in pressure is detected, the possible root causes are determined to be the access device distal kink (2) or the kinked sampling line and access device (3) (block 910). The determination for possible root causes, in some embodiments, is based on an algorithm for detecting occlusions.

As illustrated at block 912, the sampling line is flushed. For example, the flow module system 500 or 502 may be used to flush the sampling line with IV solution. In response to flushing the sampling line, low compliance or high compliance is detected as illustrated at blocks 914 and 920, respectively. When low compliance is detected, the likely root cause of the failed blood sample is determined to be the kinked sample line and access device as illustrated at block 916. As illustrated in block 918, the system is stopped and user intervention is requested. For example, the monitor 12 may display an error message, a failed blood sample code, or instructions that the access device and/or sampling line should be adjusted or replaced. In other examples, an audio or tactile signal such as a beep or vibration may be emitted by the monitor 12 or some other device in communication with the glucose sensing system 10.

When high compliance is detected (block 920), the likely root cause is determined to be the access device distal kink (2) as illustrated at block 922. As further illustrated in block 924, the system is stopped and user intervention is requested such as adjustment and/or replacement of the access device.

Referring again to block 926, normal pressure is detected when the jacket is flushed. As illustrated at block 928, the possible root causes are determined to be the access device wall contact (1), the clot in internal lumen of sample line (4) and the clot hanging on tip of sampling line (5) when normal pressure is detected. In response to determining normal pressure, the sampling line is flushed as illustrated at block 930. When the sample line is flushed, increased pressure or normal pressure is detected as illustrated in blocks 932 and 938, respectively.

As illustrated at block 934, the likely root cause is determined to be the clot in internal lumen and sampling line (4) when increased pressure is detected. In response to this determination, a high pressure sampling line flush is performed to dislodge the clot and/or the system is stopped and user intervention is requested. For example, if the high pressure flushing does not unclog the sampling line, the user may be notified to replace the sample line. In some cases, if high pressure sampling is performed a predetermined number of times previously (e.g., 10 times in the last 72 hours), the user may be notified that the reference sampling line should be replaced. In such cases, the repeated clotting and flushing of the sampling line may have caused damage to the sampling line such that a replacement is required.

Referring again to block 938, normal pressure is detected when the sampling line is flushed. As illustrated at block 940, the possible root causes are determined to be the access device wall contact (1) and clot hanging on tip of sampling line (5) when normal pressure is detected. In response to the determination, fluid is drawn through the jacket and/or sampling line as illustrated at block 942.

When fluid is drawn through the jacket and/or sampling line, increased pressure or normal pressure is detected as illustrated at blocks 944 and 950, respectively. As illustrated at block 946, the likely root cause is determined to be the access device wall contact (1) when increased pressure is detected. To correct the problem, the system is stopped and user intervention is requested such as a request to adjust the position of the access device (block 948).

As illustrated at block 952, the likely root cause for the failed blood sample is determined to be the clot hanging on tip of sampling line (5) when normal pressure is detected (see block 950). In response to the determination of the root cause, simultaneous high pressure flushes of the sampling line and jacket are performed to dislodge the clot and/or the system is stopped and user intervention is requested (replace sampling line) as illustrated at block 954.

There are many variants of the algorithm described herein. The respective draws and flushes can be placed in a variety of different orders, and if more root causes of failed blood sampling are identified, these could be incorporated into the analysis and correction algorithm. In addition, diagnostic information could also be gathered during blood draws. One example of this would be to measure the pressures during blood draws. In situations of failed blood samples in which an increased blood draw pressure was observed, the flowchart illustrated in FIG. 9 could be followed. However, if a failed blood draw (such as identified by the non-enzyme electrode) displayed normal or low pressure readings (such as might occur if a vein with low flow caused dilution of the current blood sample with the last calibration flush), a separate diagnostic algorithm could be used, or the user could be alerted directly that a low-flow vein was the most likely root cause of the difficulty. This algorithm could also be combined with existing algorithm capabilities, such as the current ability of the glucose monitoring system to signal a failed blood sample due to signal noise (usually due to ESL) interference or patient movement).

FIG. 10 illustrates a system and environment 1000 for analyzing and correcting failed blood sampling. In the environment 1000, the flow controller 20, the monitor 12, and a user 1002 is provided. As shown in the illustrated embodiment, the flow controller 20 and/or pressure sensor 1010 is in communication with the monitor 12. The flow controller 20 and/or pressure sensor 1010 may be operably connected to the monitor 12 using electrical connectors, near field communication, wireless technology, and the like. The pressure sensor 1010, as shown in FIG. 10, is associated with the flow controller 20. The pressure sensor 1010 may be, for example incorporated into the flow controller 20, externally attached to a mounting bracket on the pole 34, or associated with the external tubing of the flow controller 20.

The monitor 12, in the exemplary embodiment, includes various features, such as a communication interface 1012, a processing device 1014, a user interface 1024, and a memory device 1016. The communication interface 1012 includes a device that allows the monitor 12 to communicate over a network (not shown).

As used herein, a “processing device,” such as the processing device 1014, generally refers to a device or combination of devices having circuitry used for implementing the communication and/or logic functions of a particular system. For example, a processing device may include a digital signal processor device, a microprocessor device, and various analog-to-digital converters, digital-to-analog converters, and other support circuits and/or combinations of the foregoing. Control and signal processing functions of the system are allocated between these processing devices according to their respective capabilities. The processing device 1014 may further include functionality to operate one or more software programs based on computer-executable program code thereof, which may be stored in a memory. As the phrase is used herein, a processing device 1014 may be “configured to” perform a certain function in a variety of ways, including, for example, by having one or more general-purpose circuits perform the function by executing particular computer-executable program code embodied in computer-readable medium, and/or by having one or more application-specific circuits perform the function.

The processing device 1014 is also configured to access the memory device 1016 in order to read the computer readable instructions 1018, which in some embodiments include a bodily fluid sample analysis application 1020. The memory device 1016 also may have a datastore 1022 or database for storing pieces of data for access by the processing device 1014.

As used herein, a “user interface” 1024 generally includes a plurality of interface devices that allow the user 1002 to input commands and data to direct the processing device 1014 to execute instructions. As such, the user interface 1024 employs certain input and output devices to input data, such as patient data, or output data. These input and output devices may include a display, mouse, keyboard, button, touchpad, touch screen, microphone, speaker, LED, light, joystick, switch, buzzer, bell, and/or other user input/output device for communicating with one or more users or systems.

As used herein, a “memory device” 1016 generally refers to a device or combination of devices that store one or more forms of computer-readable media and/or computer-executable program code/instructions. For example, in one embodiment, the memory device 1016 includes any computer memory that provides an actual or virtual space to temporarily or permanently store data and/or commands provided to the processing device 1014 when it carries out its functions described herein.

As shown in FIG. 11, an exemplary flow profile of one embodiment of the present disclosure includes a calibration and flush phase of about 276 seconds which includes 3.2 mL/hr for calibration, a flush of 650 mL/hr and trailing rates of 1.9 mL/hr and zero flow for a short time period. In the draw and sample phase, a 3.5 mL/hr draw is used with a zero flow rest period at the end. This is followed by the beginning of the flush phase with a 24 second “clear” flush using a 5 mL/hr start and then a ramped-up pre-calibration flush rate of 650 mL/hr.

In some embodiments, the system 10 may be employed over a 72 hour period and sample blood with 40 to 200 mL volumes in 5 to 10 minute cycles. With a 5 minute target blood glucose cycle and an approximate 90 second time window for draw volume, the maximum draw rate is about 200 mL/hour.

As will be appreciated by one skilled in the art, aspects of the present disclosure may be embodied as a system, method or computer program product. Accordingly, aspects of the present disclosure may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, aspects of the present disclosure may take the form of a computer program product embodied in one or more computer readable medium(s) having computer readable program code embodied thereon.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium may be transmitted using any appropriate medium, including but not limited to wireless, wireline, optical fiber cable, RF, etc., or any suitable combination of the foregoing.

Computer program code for carrying out operations for aspects of the present disclosure may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Aspects of the present disclosure are described below (and above) with reference to flowchart illustrations and/or block diagrams of methods, apparatus (systems) and computer program products according to embodiments of the present disclosure. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

The flowchart and block diagrams in the figures illustrate the architecture, functionality, and operation of possible implementations of systems, methods and computer program products according to various embodiments of the present disclosure. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

As is evident from the range of modeled and experimentally verified embodiments described above, the present disclosure is not to be limited to the specific embodiments disclosed, and that modifications and other embodiments are intended to be included within the scope of the appended claims. Although specific terms are employed herein, they are used in a generic and descriptive sense only and not for purposes of limitation.

That which is claimed:

1. A method for analyzing failed bodily fluid sampling, the method comprising:
providing an analyte sensing system having a sensor coupled to a sampling line and an access device configured to sample bodily fluid, the access device having an inner space, wherein the sampling line is positioned in the inner space, the analyte sensing system comprising a flow controller configured to draw bodily fluid through the sampling line and inner space of the access device and flush a fluid through the sampling line and inner space and a monitor in communication with the sensor and the flow controller, the monitor comprising a computer apparatus including a processor and a memory;
performing, via the sensing system, a sampling event of a subject's vasculature;
obtaining, continuously and/or intermittently, one or more status values of the sampling event via the sensing system;
comparing, via the sensing system, the one or more status values with at least one predetermined value corresponding to a failed sampling event; and
determining, via the sensing system, whether the sampling event failed based on the comparing step,
wherein, in the event of the sampling event failing, the method further comprising:
flushing or drawing the sampling line or a portion of the inner space of the access device via the flow controller.

2. The method of claim 1, further comprising:
detecting pressure in response to flushing or drawing the sampling line or the portion of the inner space; and
determining one or more possible root causes for the failure of the sampling event based on the detected pressure.

3. The method of claim 2, further comprising:
in response to determining the one or more possible root causes, terminating the sensing system, requesting user intervention, performing flushing of the sampling line, or performing flushing of the portion of the inner space.

4. The method of claim 1, further comprising:
detecting a normal pressure in response to flushing the sampling line or the portion of the inner space of the access device;
detecting an increased pressure in response to drawing the sampling line or the portion of the inner space of the access device;
determining that the failed sampling event is caused by a port of the access device being in contact with the wall of the subject's vasculature containing the access device.

5. The method of claim 4, the method further comprising:
stopping the analyte sensing system; and
requesting the user to adjust the position of the access device.

6. The method of claim 1, the method further comprising:
detecting an increased pressure in response to flushing or drawing the sampling line or the portion of the inner space of the access device;
detecting, in response to flushing the sampling line, high compliance;
determining that the failed sampling event is caused by the access device kinking at a location that is distal to the distal end of the sampling line.

7. The method of claim 1, the method further comprising:
detecting an increased pressure in response to flushing or drawing the sampling line or the portion of the inner space of the access device;
detecting, in response to flushing the sampling line, low compliance;
determining that the failed sampling event is caused by the kinking of the access device and the sampling line, the access device kinking at a location that is proximal to the distal end of the sampling line.

8. The method of claim 1, further comprising:
detecting a normal pressure in response to flushing or drawing the portion of the inner space of the access device;
detecting an increased pressure in response to flushing or drawing the sampling line;
determining that the failed sampling event is caused by a clot formed in the internal lumen of the sampling line.

9. The method of claim 8, further comprising:
performing a high pressure flush of the sampling line to dislodge the clot.

10. The method of claim 1, further comprising:
detecting a normal pressure in response to flushing or drawing the portion of the inner space of the access device;
detecting a normal pressure in response to flushing the sampling line;
detecting an increased pressure in response to drawing the sampling line;
determining that the failed sampling event is caused by a clot formed on the tip of the sampling line.

11. The method of claim 10, further comprising:
performing, simultaneously, a high pressure flush of the sampling line and the portion of the inner space of the access device to dislodge the clot.

12. A method comprising:
providing an analyte sensing system having a sensor coupled to a sampling line and an access device configured to sample bodily fluid, the access device having an inner space, wherein the sampling line is positioned in the inner space, the analyte sensing system comprising a flow controller configured to draw bodily fluid through the sampling line and inner space of the access device and flush a fluid through the sampling line and inner space and a monitor in communication with the sensor and the flow controller, the monitor comprising a computer apparatus including a processor and a memory;
performing, via the sensing system, a sampling event of a subject's vasculature;
obtaining, continuously and/or intermittently, one or more status values of the sampling event via the sensing system;
comparing, via the sensing system, the one or more status values with at least one predetermined value corresponding to a failed sampling event; and
determining, via the sensing system, whether the sampling event failed based on the comparing step,
wherein in the event of the sampling event failing, the method further comprising:
flushing or drawing the sampling line or a portion of the inner space of the access device via the flow controller;
detecting pressure in response to flushing or drawing the sampling line or the portion of the inner space; and
determining one or more possible root causes for the failure of the sampling event based on the detected pressure.

13. The system of claim 12, wherein the one or more possible root causes for the failure of the sampling event comprises at least one of (i) a port of the access device contacts the wall of the subject's vasculature containing the access device; (ii) the access device kinks at a location that is distal to the distal end of the sampling line; (iii) the access device kinks at a location that is proximal to the distal end of the sampling line; (iv) a clot forms in the internal lumen of the sampling line; and (iv) a hanging clot forms on the tip of the sampling line.

14. The method of claim 12, further comprising:
in response to determining the one or more possible root causes, terminating the sensing system, requesting user intervention, performing flushing of the sampling line, or performing flushing of the portion of the inner space.

15. A system for analyzing failed bodily fluid sampling, the system comprising:
a sensor coupled to a sampling line and an access device configured to sample bodily fluid, the access device having an inner space, wherein the sampling line is positioned in the inner space;
a flow control system configured to draw bodily fluid through the sampling line and inner space of the access device and flush a fluid through the sampling line and inner space; and
a monitor in communication with the sensor and flow control system, the monitor comprising a computer apparatus including a processor and a memory; and
a bodily fluid sample analysis module stored in the memory, comprising executable instructions that when executed by the processor cause the processor to:
obtain, continuously and/or intermittently, one or more status values of a sampling event;
compare the one or more status values with at least one predetermined value corresponding to a failed sampling event; and
determine whether the sampling event failed based on the comparing step;
cause the flow control system to flush or draw the sampling line or a portion of the inner space of the access device.

16. The system of claim 15, wherein the module is further configured to:
cause the sensor to detect pressure in response to flushing or drawing the portion of the inner space of the access device or the sampling line; and
determine one or more possible root causes for the failure of the sample based on the detected pressure.

17. The system of claim 16, wherein the module is further configured to:
in response to determining the one or more possible root causes, terminate the sensing system, request user intervention, perform flushing of the sampling line, or perform flushing of the portion of the inner space.

18. The system of claim 15, wherein the module is further configured to:
detect a normal pressure in response to flushing the sampling line or the portion of the inner space of the access device;
detect an increased pressure in response to drawing the sampling line or the portion of the inner space of the access device;
determine that the failed sampling event is caused by a port of the access device being in contact with the wall of the subject's vasculature containing the access device.

19. The system of claim 15, wherein the module is further configured to:
detect an increased pressure in response to flushing or drawing the sampling line or the portion of the inner space of the access device;
detect, in response to flushing the sampling line, high compliance;
determine that the failed sampling event is caused by the access device kinking at a location that is distal to the distal end of the sampling line.

20. The system of claim 15, wherein the module is further configured to:
detect an increased pressure in response to flushing or drawing the sampling line or the portion of the inner space of the access device;
detect, in response to flushing the sampling line, low compliance;
determine that the failed sampling event is caused by the kinking of the access device and the sampling line, the access device kinking at a location that is proximal to the distal end of the sampling line.

21. The system of claim 15, wherein the module is further configured to:
detect a normal pressure in response to flushing or drawing the portion of the inner space of the access device;
detect an increased pressure in response to flushing or drawing the sampling line;
determine that the failed sampling event is caused by a clot formed in the internal lumen of the sampling line.

22. The system of claim 15, wherein the module is further configured to:
detect a normal pressure in response to flushing or drawing the portion of the inner space of the access device;
detect a normal pressure in response to flushing the sampling line;
detect an increased pressure in response to drawing the sampling line;
determine that the failed sampling event is caused by a clot formed on the tip of the sampling line.
